(19) United States
(12) Patent Application Publication
(10) Pub. No.: US 2014/0087396 A1
(43) Pub. Date: Mar. 27, 2014

(54) Health Diagnostic Compact Disc

(71) Applicant: THE BOARD OF TRUSTEES OF THE UNIVERSITY OF ILLINOIS, Urbana, IL (US)

(72) Inventors: Syed M. Imaad, Lahore (PK); Gang Logan Liu, Champaign, IL (US)

(21) Appl. No.: 13/881,828

(22) PCT Filed: Oct. 27, 2011
(86) PCT No.: PCT/US2011/058079

§ 371 Date: Dec. 5, 2013

Related U.S. Application Data
(60) Provisional application No. 61/407,201, filed on Oct. 27, 2010.

(51) Int. Cl. G01N 33/543; G11B 7/2403

(52) U.S. Cl. CPC G01N 33/54386; G11B 7/2403
USPC 435/7.92; 428/64.7; 436/501; 435/7.1; 435/39; 435/19; 435/15

(57) ABSTRACT

A modified health diagnostic compact disc (HDCD) comprising (a) a protective layer; (b) a reflective layer; (c) a dye layer (d) a polycarbonate layer; and (e) a micro fluidic layer. In a certain aspect, the polycarbonate layer of the HDCD contains a data layer. The micro fluidic layer of the HDCD is composed of polydimethylsiloxane (PDMS). The micro fluidic layer of the HDCD contains one or more donut-shaped trenches. The micro fluidic layer of the HDCD is associated with conjugated microparticles in solution. The HDCD further comprises integrated microneedles. The modified health diagnostic compact disc can be used by methods of the invention to identify and/or measure the presence of biologic cells utilizing a standard computer compact disk drive.

### CROSS-REFERENCE TO RELATED APPLICATIONS

This application claims benefit of U.S. Provisional Application No. 61/407,201 filed Oct. 27, 2010, which is incorporated herein by reference in its entirety.

### GOVERNMENT RIGHTS

This invention was made with government support under contract number CIMIT-C5769 awarded by the Center for Integration of Medicine & Innovative Technology. The government has certain rights in the invention.

### FIELD OF THE INVENTION

This invention provides diagnostic methods and apparatus for identifying and measuring biological cells. More particularly, the invention provides methods and reagents for producing a microfluidic compact disc that can be utilized with a standard computer compact disk drive to identify biological cells in a patient sample. Certain embodiments of the invention provide a health diagnostic compact disk (HDCD), wherein said HDCD comprises a microfluidic layer for applying a patient cell sample. HDCDs are used according to methods disclosed herein to identify the presence of cells, in particular red blood cells. This invention further provides methods and apparatus for diagnosing leukemia, anemia, Gibson syndrome, COPD, sickle cell disease, internal bleeding, fever, chronic inflammation, heart disease, heart attack, myoinfarction, issues related to liver function, coronary heart disease, septics or acquired immunodeficiency disorder (AIDS), for example. Particularly advantageous is the ability to identify and measure patient cells applied to the HDCD with a standard computer compact disk drive, thus eliminating the need for expensive or sophisticated diagnosis machinery. This greatly expands availability of diagnostic tools to underprivileged populations.

### BACKGROUND OF THE INVENTION

According to United Nations estimates, nearly half of the world's population lives in rural areas. The remote location of these areas has denied its residents the access to quality health care such as basic medical facilities like those usually found in urban centers. Failure to detect and monitor disease rapidly and accurately often results in detrimental epidemics. Providing rapid, affordable and point-of-care diagnostics, therefore, is vital for improving global health [1]. This has sparked a tremendous increase the development of portable microfluidic diagnostic tools over the past decade [2-11].

Microfluidic devices offer a number of advantages including the use of low sample volumes (on the order of few microliters down to nanoliters), rapid results, and greater portability. Additionally, devices and systems can be designed such that minimal operator experience or training is required. This opens up a large range of possibilities for individuals to check their health conditions. For instance, immuno-chromatographic strips often used to test for sexually transmitted diseases and pregnancy are examples of qualitative diagnostic tools that provide users with crude results in the form of a “yes” or “no”, but not “how”. However more quantitative analysis, even with using microfluidic devices, often requires expensive and bulky instruments such as confocal microscopy systems and electronic spectrum analyzers.

In sharp contrast to the above exclusively scientific instruments, digital compact disc (CD), CD drives and personal computers are highly ubiquitous and affordable commodities worldwide, not only available to the affluent residents of urban centers in the developed nations but are also finding their way into villages and other remote regions of the world, providing access to the underprivileged in the developing and developed world. Incorporation of microfluidic capabilities into a conventional data or music CD and blending of quantitative liquid information with the binary data recorded on CD, such that it could still be detected by a standard CD drive, many would greatly benefit from the merits of both technologies for global health applications.

The health diagnostics compact disc (HDCD) device of the present invention has the potential of providing low-cost, portable, point-of-care quantitative diagnostics by replacing bench-based devices and techniques. In essence, the invention comprises a transformed CD—an inexpensive plastic data storage medium that has been converted into a microfluidic health diagnostic device (see FIG. 1). An innovative method of detecting and measuring biomolecules and cells using digital data media facilitates the conversion of biological information into digital information. The focused laser beam illuminated and reflected on the data layer of the HDCD is interfered with by microparticles, cells or biomolecules in the microfluidics layer which is directly above the data layer. The original digital information is hence changed due to this interference and the alteration directly correlates with the shape, concentration, optical density and many other properties of the microparticles, cells or biomolecules. The HDCD of the present invention is designed to be used in the same way as a standard music or data CD would be, thus making self-diagnosis at home possible by using personal computers with standard CD drives.

Previous work on the use of CD based solutions for biomolecular detection has focused on two different, basic approaches: (1) surface chemistry on conventional CD surface and (2) flow regulation (and surface chemistry) with CD-like microfluidic platform.

Regarding the surface chemistry approach, Yu et al. [12] have reported DNA immobilization and detection on a polycarbonate (PC) surface by chemically modifying the surface using UV/ozone. In a more recent report, standard CD drives were used to detect the DNA binding by metal nanoparticle staining on CD surface [13]. The data errors due to nanoparticle scattering on the CD surface are reported by the CD drive and are therefore interpreted as DNA binding. La Clair and Burkat [14] have used a similar approach by employing error rate analysis to detect different ligands and biomolecules that were attached to the surface of a conventional CD and read using standard CD drives. Large concentrations of biomolecules imply greater binding and hence higher error rate picked up from the CD drive. Maquieira et al. [15] have used conventional CDs, but modified CD drives, to bind and detect low-abundant compounds that are commonly used as pesticides.

Regarding the flow regulation approach, Madou et al. have pioneered on the use of CD-like microfluidic platforms for various biosensing applications such as carrying out an enzyme linked immunosorbent assay (i.e., ELISA) [16] and the fluid flow is regulated by varying the speed of rotation of the disc. However detection is carried out using a high-end fluorescence microscopy system, not a standard CD drive on personal computers. Ducree et al. [17] have also demonstrated the use of a CD-like microfluidic disc for applications that include volume metering, volume splitting, mixing and routing but again a separate optical detection system is used in conjunction.

For all previous work with standard CD drives, microfluidics has not been employed with a CD. In addition all tests have only been performed on the top surface of the CD hence requiring laborious surface chemistry protocols and bearing with low spatial resolution. Furthermore, all existing CD-like microfluidic platforms require separate detection units that ultimately turn out to be expensive and non-portable solutions. In comparison, for the first time, the present invention successfully demonstrates the direct integration of microfluidics with conventional CDs while using a standard CD drive in personal computer as the detection instrument. The present invention eliminates the need for separate detection units and tedious surface treatment process.

Cell counting and determination of various cell types are issues of immense biomedical significance and often microscopy or flow cytometry system has to be used. Blood cell counting and sizing are often standard medical practices in the diagnosis of many other diseases such as leukemia, anemia, septics, and acquired immunodeficiency syndrome (AIDS) etc. Herein is the first disclosure of a CD based microfluidic device (the HDCD) compatible with standard CD drives/readers and use of a digital microfluidic CD for detection of various concentrations of microparticles, cells and biomolecules. This HDCD opens a new door for the integration of polymer microfluidics with conventional CDs in such a way that standard CD drives can be used as the reading instrument for biomolecule and cell detections.

For fabrication of the device, throughput can be significantly increased and cost can be further reduced if injection molding process is used to make CDs with polycarbonate microfluidic layer, thus eliminating the use of polydimethylsiloxane (PDMS) and clean room facilities. Despite the cost of development of our prototype being relatively low, the fact that a single device can be reused (by detaching, cleaning, and re-bonding PDMS microfluidic layer) will reduce costs further in the long run. This reduced cost will potentially lower expenditures associated with primary healthcare while at the same time make home-medicine and tele-medicine possible. With the fully developed microfluidic CD device all that is required for molecular and cellular diagnosis is this device and a standard CD drive on a desktop or laptop computer, with minimal sample preparation and handling. The device can greatly benefit the healthcare practices in resource-limited settings such as remote senior residences in the U.S. and poor villages in the developing world.

### SUMMARY OF THE INVENTION

In one aspect, the invention is a health diagnostic compact disc (HDCD) comprising: (a) a protective layer; (b) a reflective layer; (c) a dye layer (d) a polycarbonate layer; and (e) a microfluidic layer. In a certain aspect, the polycarbonate layer of the HDCD contains a data layer. In another aspect, the microfluidic layer of the HDCD is composed of polydimethylsiloxane (PDMS). In yet another aspect, the microfluidic layer of the HDCD contains one or more donut-shaped trenches. In a further aspect, the microfluidic layer of the HDCD is associated with conjugated microparticles in solution. In another aspect, the HDCD further comprises integrated microneedles.

In one aspect, the invention is a method for detecting the presence of biomolecules or cells comprising: (a) loading the microfluidic layer of the HDCD of claim 1 with conjugated microparticles in solution; (b) incubating the HDCD to allow for microparticles to immobilize to the polycarbonate layer; (c) using a standard compact disc (CD) drive to establish a baseline reading; (d) loading a biomolecule or cell solution to the into the microfluidic layer of the HDCD; (e) incubating the HDCD to allow for interaction of the biomolecule or cell solution with the immobilized microparticles; (f) using a standard CD drive to obtain error rates; and (e) comparing the data generated in steps (c) and (f) for determining a diagnosis. In another aspect, the diagnosis comprises cell counting, detecting biomolecule or cell concentration, detecting cell type, or detecting biomolecular binding. In a certain aspect, biomolecular binding using the HDCD is via an ELISA. In another aspect, cell counting using the HDCD comprises counting of red blood cells for diagnosing leukemia, anemia, Gibson syndrome, COPD, sickle cell disease, internal bleeding, fever, chronic inflammation, heart disease, heart attack, myoinfarction, issues related to liver function, coronary heart disease, septics or acquired immunodeficiency disorder (AIDS). In yet another aspect, cell counting using the HDCD comprises counting of red blood cells for determining effectiveness of chemotherapy, athletic ability or athletic stamina.

In another aspect, the invention comprises a kit for detection of biomolecules or cells comprising the HDCD of the invention and software for use in a personal computer which generates a diagnostic report of the detected biomolecules or cells.

### BRIEF DESCRIPTION OF THE DRAWINGS OF THE INVENTION

FIG. 1 shows a cross-sectional configuration of a digital microfluidic compact disc.

FIG. 2 shows a schematic representation of the soft-lithography process for polydimethylsiloxane (PDMS) microfluidic layer.

FIG. 3 shows a schematic representation of the fabrication process for bonding PDMS microfluidic layer to CD.

FIG. 4 shows a diagram of the “Active” microfluidic layer of the HDCD.

FIG. 5 shows detections of immobilized nanoparticles in microfluidic CD. FIG. 5(a) shows a data block error rate picked up from the microfluidic CD with empty microfluidic channel. FIG. 5(b) shows a data block error rate picked from the microfluidic CD with the channel partially loaded with particles.

FIG. 6 shows concentration measurement of 10 μm microparticle solution in microfluidic CD. FIG. 6(a) shows the shown data error rates picked up from the microfluidic channel filled with 25% concentration; FIG. 6(b) shows the shown data error rates picked up from the microfluidic channel filled with 50% concentration, and FIG. 6(c) shows the shown data error rates picked up from the microfluidic channel filled with 100% concentration of microparticle solution. FIG. 6(d) shows data block error rates as the function of micro particle concentration.

FIG. 7 shows the concentration measurement of living cell solutions in microfluidic CD. FIG. 7(a) shows the shown data error rates were picked up from when the microfluidic channel is loaded with zero cells; FIG. 7(b) shows the shown data error rates were picked up from when the microfluidic channel is loaded with 1×10⁶ cells/mL; and FIG. 7(c) shows the shown data error rates were picked up from when the microfluidic channel is loaded with 9×10⁶ cells/mL of CHO cells. FIG. 7(d) shows data block error rates as the function of cell concentration.

FIG. 8 shows a schematic for ELISA on the HDCD.

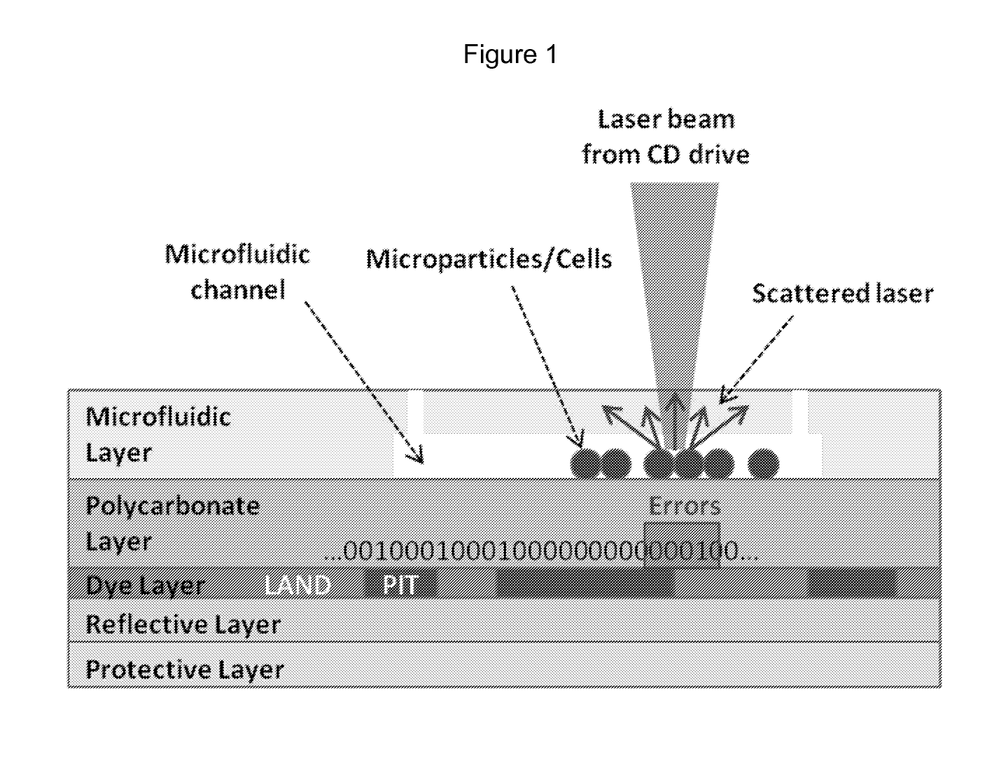

### DETAILED DESCRIPTION OF THE INVENTION

In one aspect the invention is a health diagnostic compact disc (HDCD) comprising: (a) a protective layer; (b) a reflective layer; (c) a dye layer; (d) a polycarbonate layer; and (e) a microfluidic layer, and is derived from a standard CD-R music or data storage media. As is with all standard CD-R music or data storage media, the protective layer is composed of plastic, the reflective layer is metallic, and the dye layer comprises a photosensitive material. The thin polycarbonate layer contains data added, which provides a baseline output for the method envisioned by the invention. In one aspect, this data can be binary data. In another aspect, the microfluidic layer is composed of polydimethylsiloxane (PDMS). In a further aspect, the microfluidic layer contains one or more donut-shaped trenches. The inner diameter of the trenches can be 3.0-4.0 cm, and the outer diameter of the trenches can be 7.0-8.0 cm in diameter. Furthermore, the trenches can be machined up to 75% of the total thickness of the HDCD. In yet another aspect, the microfluidic layer contains conjugated microparticles in solution. The microparticles can be composed of polystyrene, silica, gelatin, or polycarbonate. In certain embodiments, the microparticles are conjugated with glutaraldehyde, biotin, streptavidin, DNA, peptides, or antibodies.

In one aspect, the HDCD of the invention further comprises integrated microneedles and multiplexed microfluidic network. It is envisioned that this HDCD can be used for mobile blood analysis. Also contemplated by the invention are standalone and multifunctional microfluidic CD which utilizing the centrifugal force in CD spinning for molecule and cell separation. A patient in need of a diagnosis can press their fingertip on the HDCD and extract blood via the microneedles. Microneedles are integrated on the HDCD and connected to the microfluidic inlet. Blood streams collected by the microneedles can be introduced into the connected microfluidic channels.

They can then place the HDCD into a computer optical drive, which then facilitates microfluidic cell separating and capturing, as well as cell counting, imaging, and molecular detecting on spinning HDCD. The cell sorting envisioned by the inventors can separate red blood cells from white blood cells. The personal computer housing the optical drive contains software that can perform digital data analysis and cell image reconstruction. The resulting data is then transmitted to a healthcare provider via the internet or a wireless network. Utilization of surface chemistry on the HDCD is also envisioned.

In a particular aspect, the invention is a method for detecting the presence of biomolecules or cells comprising: (a) loading the microfluidic layer of the HDCD of the invention with conjugated microparticles in solution; (b) incubating the HDCD to allow for microparticles to immobilize to the polycarbonate layer; (c) using a standard compact disc (CD) drive to establish a baseline reading; (d) loading a biomolecule or cell solution to the into the microfluidic layer of the HDCD; (e) incubating the HDCD to allow for interaction of the biomolecule or cell solution with the immobilized microparticles; (f) using a standard CD drive to obtain error rates; and (e) comparing the data generated in steps (c) and (f) to determine a diagnosis.

As used herein, the term “cell” can be any eukaryotic cell from any patient, including any vertebrate animal, in need of medical diagnosis. As used herein, the term “biomolecule” can be any molecule that exists in the body of a patient. These can be, for example, amino acids, nucleic acids or polypeptide chains such as antibodies.

In aspects of the invention, the baseline readings and the error rates are represented as graphs. These graphs can specifically be of the block error rate (BLER), which is a measure of the total count of errors encountered in a section of the disc. The physical principle is that biomolecular binding (labeled using blue microspheres) or the introduction of microparticles or cells in the microfluidic CD device, will generate errors proportional to the concentration of biomolecules, particles or cells. Also envisioned by the invention is another type of error rate, the E22, which represents the number of double errors for the second data parity level, and can potentially provide detection based on orders of magnitude differences. The BLER can then be used for finer resolution microparticle concentration quantification within the same order of magnitude.

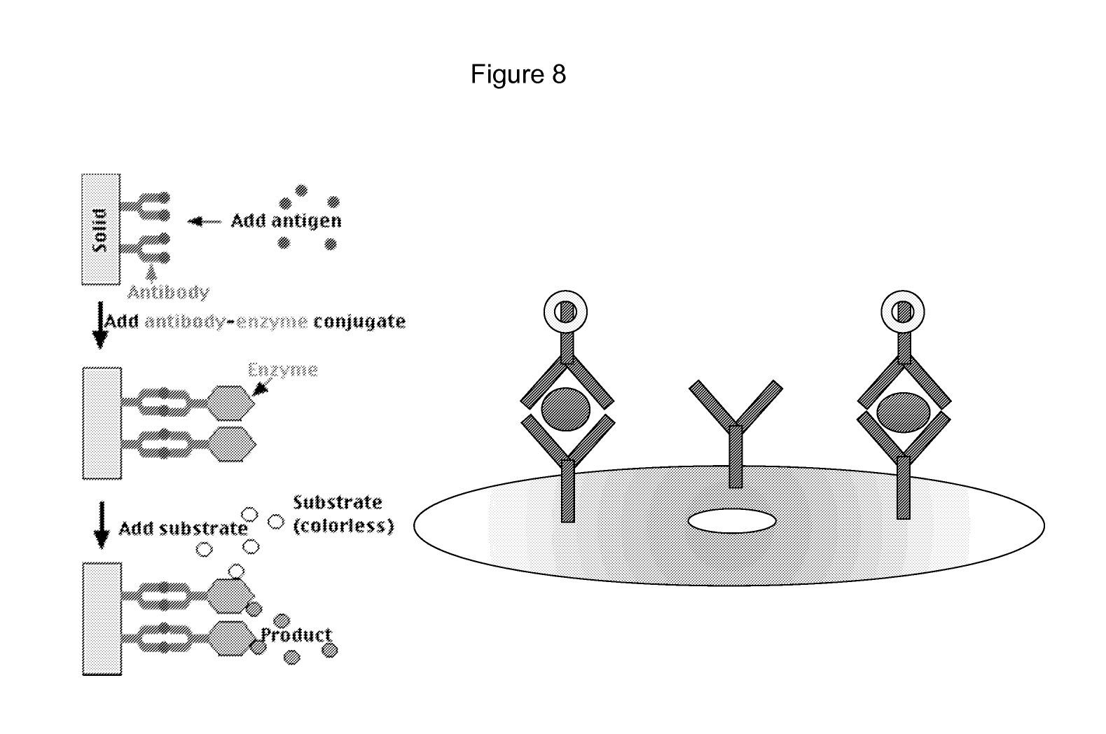

In certain aspects, the method the invention can be used for cell counting, determination of biomolecule or cell concentration, determination of cell type, or biomolecular binding. In a particular aspect, the biomolecular binding is used in ELISA (see FIG. 8).

In aspects of the invention, the method can be used for cell counting of red blood cells and diagnosis of leukemia, anemia, Gibson syndrome, COPD, sickle cell disease, internal bleeding, fever, chronic inflammation, heart disease, heart attack, myoinfarction, issues related to liver function, coronary heart disease, septics or acquired immunodeficiency disorder (AIDS). In further aspects, cell counting can be used for counting of red blood cells and determination of effectiveness of chemotherapy, athletic ability or athletic stamina. The following specific blood-related tests are contemplated by the instant invention:
(1) testing red blood cell count to determine concentration for diagnosis of anemia;
(2) testing hemoglobin to determine optical density of red blood cells for diagnosis of oxygen levels related to Gibson syndrome;
(3) testing hemocrit to determine red blood cell proportion for diagnosis of COPD or determination of athletic ability;
(4) testing red blood cell shape for diagnosis of sickle cell disease;
(5) testing platelet count to determine platelet concentration for diagnosis of thrombocytosis;
(6) testing neutrophile count to determine neutrophile concentration for diagnosis of internal bleeding;
(7) testing lymphocyte count to determine lymphocyte concentration for diagnosis of leukemia, effectiveness of chemotherapy, or HIV;
(8) testing monocyte count to determine monocyte concentration for diagnosis of fever or chronic inflammation;
(9) testing triglyceride-fatty acid levels using a lipase probe for diagnosis of heart disease or determination of athletic stamina;
(10) testing creatinine levels using a creatinine antibody for diagnosis of a heart attack or myoinfarction;
(11) testing alanine amino transferase and/or aspartate amino transferase levels using a alanine amino transferase and/or aspartate amino transferase antibody for diagnosis of issues related to liver function; and
(12) testing cholesterol levels using a cholesterol antibody for diagnosis of coronary heart disease.

In an additional aspect, the invention is a kit for detection of biomolecules or cells comprising an HDCD and software for use in a personal computer which generates a diagnostic report of the detected biomolecules or cells. The data generated may then be transmitted to a health care provider over the internet or a wireless network.

Other features and advantages of the invention will be apparent from the following Examples. The following are provided for exemplification purposes only and are not intended to limit the scope of the invention described in broad terms above. All references cited in this disclosure are incorporated herein by reference.

### EXAMPLES

### Example 1

### Preparation of the HDCD

The general scheme for preparation and use of the HDCD is as follows: the HDCD is derived from a standard CD-R music or data storage media and consisted of five layers including PDMS microfluidic layer, thin polycarbonate layer, photosensitive dye layer or data layer, metallic reflective layer and plastic protective layer. Microparticles, cells or biomolecules are introduced into the microfluidic channels will interfere with the converging laser beam in the optical pickup apparatus of a standard CD drive. The interference from microparticles, cells or biomolecules will cause errors in reading and decoding the digital data previously burn on the dye layer. The data errors are detected, analyzed and correlated with the particle properties in the microfluidic channel.

### (a) Writing Specific Data (Bit Sequence) to the CD

As the sequence with smallest possible constant data length should have a higher probability or “sensitivity” of being corrupted by a microparticle or cell interfering with a laser's illumination and reflection, the inventors wrote a binary data layer of “100100100 . . . ” to the compact disc. The corresponding repeating output data sequence after taking into account encoding schemes in the CD drive was used to construct a .wav audio file that repeated for half-length of a CD and the .wav file was burned to a standard band CD-R using CDBurnerXP® by software by Microsoft. Essentially this data layer served as the detection region of the device where the presence of microparticles, cells or biomolecules could be detected based on the errors generated in the data pickup process.

### (b) CD Machining and Surface Preparation

Direct integration of a thin polydimethylsiloxane (PDMS) microfluidic layer on the conventional CD surface led to large background errors and most of the times the CD was not detected at all by the CD drive. To solve this problem, the inventors used a computer numerically controlled (CNC) lathe machine to create donut shaped trenches with inner and outer diameters of 3.8 cm and 7.6 cm respectively on the polycarbonate layer of the CD, so as to cover the entire data region. The microfluidic layer would then be integrated with these trenches. The trenches we machined were 0.9 mm deep (75% of the total thickness of a normal CD).

In order to create a transparent finish similar to the original surface of the CD the rough machined CD surface was wet sanded using 15 μm and 9 μm microfinishing films, one after the other while keeping the surface wet with ethanol at all times. Wet sanding was followed by the application of plastic polish solution (Novus 2®, Tap Plastics) using microfiber cleaning cloth to increase transparency and restore the surface smoothness of the machined area.

### (c) Fabrication of Microfluidic Layer

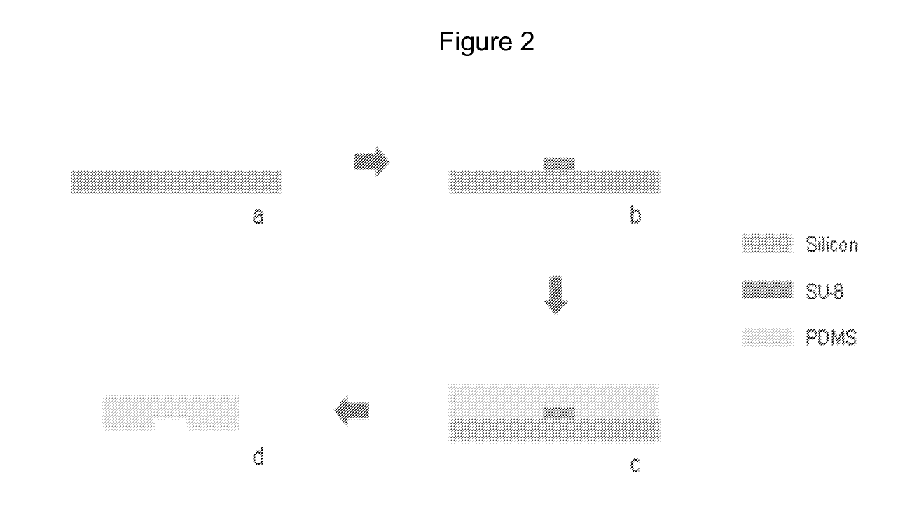

The microfluidic layer was fabricated using a soft-lithography process. A master wafer was prepared by spin-coating a 130 μm thick SU-8 2100 (MicroChem Inc.) layer on a bare silicon wafer. SU-8 was patterned using the Quintel Aligner exposure system. PDMS (10:1 v/v base-to-catalyst) was cast against the master and left to degas overnight on a completely level tabletop, followed by thermal cure at 60° C. for 45 min to complete the cross-linking. After complete solidification, the PDMS microfluidic layer was peeled off from the master resulting in a microfluidic layer with single channel of height 130 μm. The microfluidic layer was cut to dimensions of the donut trench of the machined CD. FIG. 2 summarizes this process for creating the PDMS microfluidic mold. Inlet and outlet holes were punched in the microfluidic channel using 20 gauge Luer stub adapters (Intramedic).

### (d) Bonding of Microfluidic Layer to CD

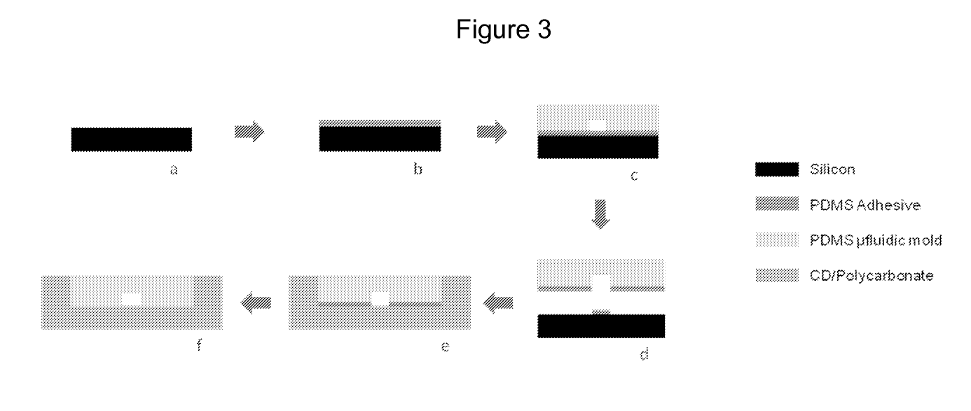

This is the most crucial step in the fabrication of the HDCD since obtaining an optically clear, bubble-free bond between the PDMS and polycarbonate surface of the CD is imperative for correct data pickup by CD drives. Usually the PDMS microfluidic mold is bonded to a glass substrate using oxygen plasma treatment of both the PDMS and glass surfaces after which the devices are bonded to give an irreversible bond. However, oxygen plasma treatment does not work with polycarbonate well. In light of these challenges the inventors devised a method as shown in FIG. 3, based on using PDMS as adhesive that not only allowed for achievement of an optically clear and bubble-free bond, but also resulted in a reversible bond allowing for detachment of the PDMS mold from the CD to reuse the device multiple times.

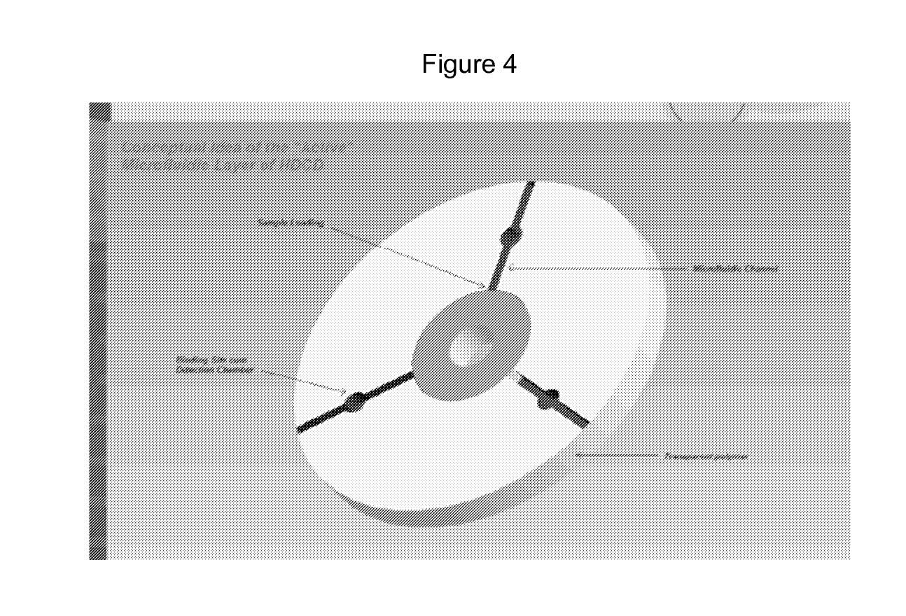

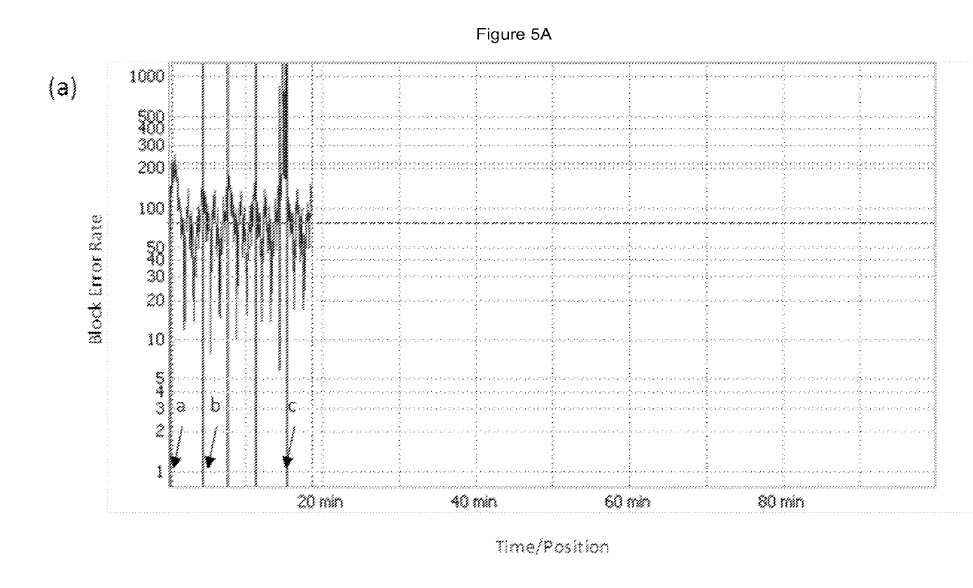

Uncured PDMS was first spin-coated on top of a 4 inch silicon wafer at 2500 rpm for 5 min to give a thickness of around 25 μm. Next, the PDMS microfluidic mold (channel side down) was placed over the PDMS coated silicon wafer to transfer PDMS adhesive to the channel side of the mold. Adhesive was not transferred to the channel since the PDMS adhesive thickness of 25 μm was much less than the channel height of 130 μm, thus ensuring that the channel was not blocked. Finally, the PDMS microfluidic mold was peeled away from the silicon wafer with PDMS adhesive and pressed against the donut trench in the CD. This was followed by a 1 hr thermal curing at 60° C. to complete the cross-linking of the PDMS adhesive, completing the bonding process. FIG. 4 shows the CD after bonding with the PDMS microfluidic mold.

### (e) Glutaraldehyde Conjugation of Microparticles

10 μm blue 500 nm polystyrene microparticles (Polysciences, Inc.) were conjugated with 8% glutaraldehyde. The conjugation protocol was as follows: (1) 0.5 ml of aqueous suspension of microspheres was placed in Eppendorf centrifuge tube; (2) the entire centrifuge tube was filled with PBS; (3) the solution was centrifuged at 10,000 rpm for 6 minutes and the supernatant was discarded; (4) the tube was then filled with PBS and shaken until the pellet was resuspended; (5) the solution was centrifuged again at 10,000 rpm for 6 minutes and the supernatant was discarded; (6) 0.5 ml of 8% glutaraldehyde in PBS was added into the tube which is then mixed well until the pellet was resuspended; (7) mixing was continued overnight on rotary shaker; (8) the solution was then centrifuged at 10,000 rpm for 6 minutes and supernatant discarded; (9) steps 4 and 5 were repeated twice; and (10) the solution tube was filled with 1 ml PBS and mixed until the pellet was resuspended.

The idea behind conjugating the particles with glutaraldehyde was to ensure binding of the particles with the exposed polycarbonate substrate in the microfluidic channel. Microparticles that were not conjugated with glutaraldehyde did not show any binding to the polycarbonate surface (see below).

### (f) Nanoparticle Attachment to Polycarbonate Surface

A 3 mm hole was punched in a PDMS piece which was bound to two polycarbonate (PC) chips derived from a CD and used as incubation well in subsequent steps for the immobilization of blue nanosphere particles to the PC surface. One PC chip was used for positive control test while the other was used for negative control test. 100 μL of glutaraldehyde modified blue nanosphere particles were added to the positive control chip, whereas unmodified blue microsphere particles were added to the negative control chip. Both were allowed to incubate overnight at room temperature. After overnight incubation, the wells were washed with PBS three times and the PC chips were blow-dried using nitrogen.

### Example 2

### Use of the HDCD

### (a) Sample Loading and Detection Using Standard CD Drive

Using the loading protocol as described above, a 10 μm glutaraldehyde conjugated blue microparticle solution of target concentration 3×10⁷ particles/mL was loaded into the microfluidic channel. The microfluidic channel was loaded using Tygon microbore PVC tubing (TGY-010, Small Parts, Inc.) and a standard 3 mL syringe with 26 gauge needle. The CD loaded with microparticle solution was then allowed to incubate at 4° C. for 2 hr. After incubation, unbound microspheres were washed away by flushing the channel with PBS solution or spinning the CD. Finally, the CD was put into a standard CD drive and the CD quality check software QpxTool was used to generate graphs of error rate against time (equivalent to position on the CD). All the experiments were run on a HP Pavilion desktop computer running Windows Vista operating software system, with a standard ATAPI DH16A6L CD drive. The CDs were read at 4× speed.

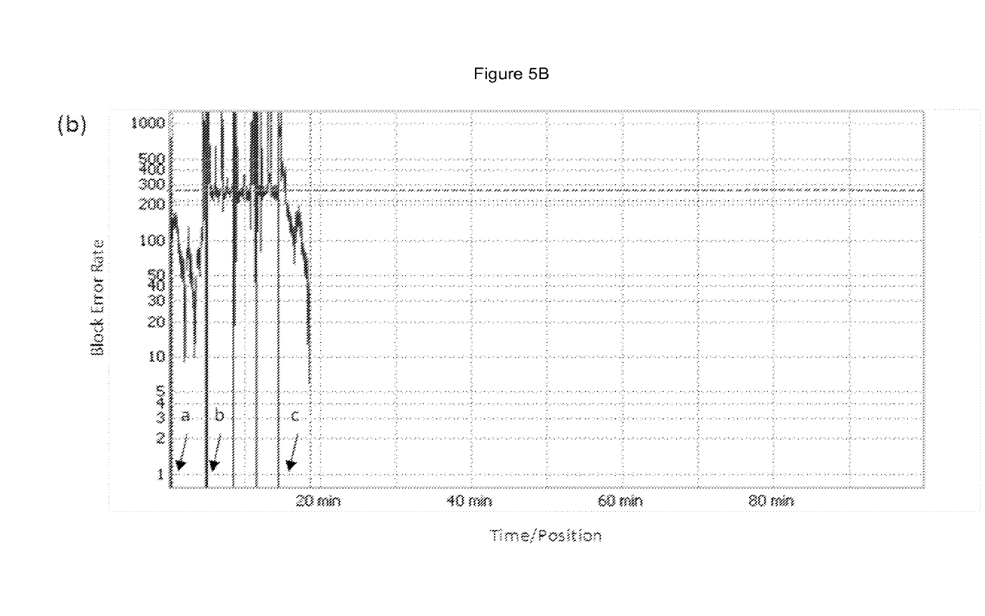

FIGS. 5(a) and 5(b) show the data block error rate with an empty microfluidic channel and with microparticles in the partial region of the microfluidic channel, respectively. Note that the total length of data burned to the CDs we used is around 20 min (compared to total CD capacity of 80 min) and hence the corresponding length of data shown on the graph is 20 min. This is true for all subsequent plots as well. Clearly a large increase in the data error rate is observed from region ‘a’ to region ‘b’ as shown in FIG. 5(b), which can be only attributed to the presence of microparticles in the channel when compared to the baseline error rate of the CD with empty microfluidic channel (FIG. 5(a)). Also it is interesting to note that at the region marked by ‘c’ i.e. the start of microfluidic channel without particles, the error rate drops back to the baseline as expected.

### (b) Detection of Microparticle Concentrations in Microfluidic CD

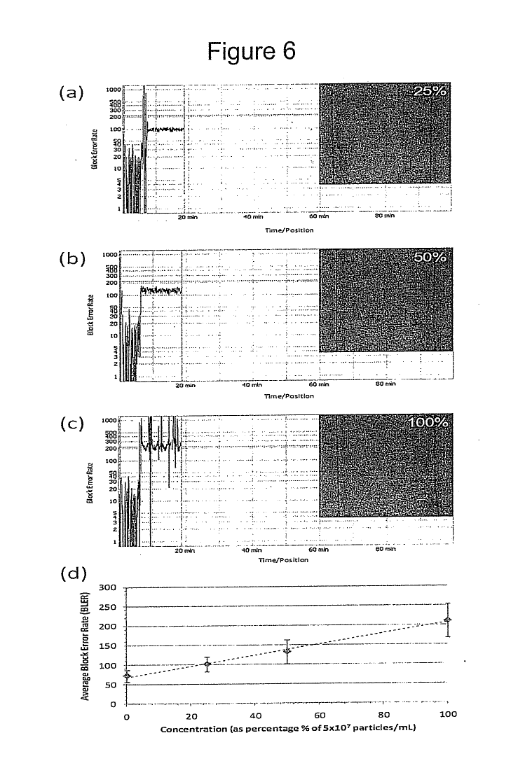

Four different concentrations of 10 μm Glutaraldehyde conjugated blue microparticle solution were loaded into the microfluidic channel of the CD and analyzed. A stock solution with the concentration of 5×10⁷ particles/mL (referred to as 100% or stock solution) was prepared. This was diluted by factors of 2 and 4 and infinitely large (pure water with no particles) to obtain solutions with 50%, 25%, 0% concentrations (with respect to stock solution) respectively. FIGS. 6 (a)-(c) show the graphs of error rate obtained.

The trend is conspicuous—an increase in the microparticle concentration leads to an increase in the error rate (FIG. 6(d)). Also note that the error rate in FIGS. 6(a)-(c) is plotted on a logarithmic scale, hence the actual differences are much larger than it appears in current plots.

### (c) Detection of Living Cell Concentration in Microfluidic CD

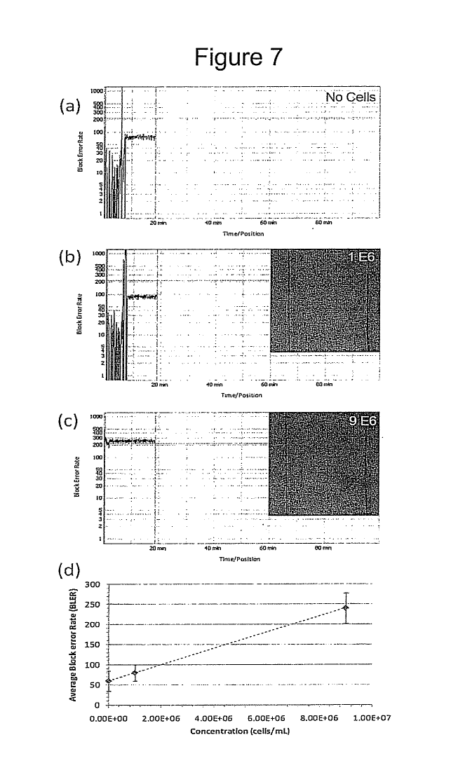

Chinese Hamster Ovarian (CHO) cells of three concentrations, 0, 1×10⁶ cells/mL and 9×10⁶ cells/mL, were successfully detected using the microfluidic CD with the lower concentration giving a lower error rate than the higher concentration. CHO cell solution was prepared in F12 medium supplemented with 10% fetal bovine serum (FBS), 1% antibioticantimycotic solution (10 units/mL penicillin G sodium, 10 μg/mL streptomycin sulfate, 25 μg/mL amphotericin B, 0.85% saline; Invitrogen, Carlsbad, Calif.), and 1% glutamine. The cell solution was introduced into the microfluidic channel of the CD and allowed to incubate at 37° C. for 2 hours. The cells were fixated by flowing in 100% methanol into the channel followed by incubation at room temperature for 10 min. This was followed by staining the fixated cells within the microfluidic channel with 0.5% crystal violet solution in 25% methanol for 10 min. The cells were stained due to the transparent nature of cells, so that a significant amount of laser scattering and hence errors could be generated. Finally, the channel was flushed with DI water multiple times and the channel left to dry. The CD's were then inserted into the CD drive to obtain error rates. FIGS. 7(a)-(c) show graphs of the error rate obtained. The error rates are clearly dependent on the cell concentrations (FIG. 7(d)). Cell counting with finer concentration resolution is certainly viable in the future as indicated by the results presented here. While only the proof-of-concept experiments were demonstrate here, more systematic work on counting living cells of various types and statuses on digital microfluidic CD is ongoing.

### REFERENCES

[1] P. Yager, T. Edwards, E. Fu, K. Helton, K. Nelson, M. R. Tam, and B. H. Weigl, “Microfluidic technologies for global public health,” Nature, vol. 442, pp. 412-418, 2006.
[2] D. J. Beebe, G. A. Mensing and G. M. Walker, “Physics and applications of microfluidics in biology,” Annu. Rev. Biomed. Eng., vol. 4, pp. 261-286, 2002.
[3] J. Knight, “Honey, I shrunk the lab,” Nature, vol. 418, pp. 474-475, 2002.
[4] A. Y. Fu, C. Spence, A. Scherer, F. H. Arnold and S. R. Quake, “A microfabricated fluorescence-activated cell sorter,” Nat. Biotechnol., vol. 17, pp. 1109-1111, 1999.
[5] M. U. Kopp, A. J. Mello and A. Manz, “Chemical amplification: continuous-flow PCR on a chip,” Science, vol. 280, pp. 1046-1048. 1998.
[6] P. J. Lee, P. J. Hung, R. Shaw, L. Jan and L. P. Lee, “Microfluidic application-specific integrated device for monitoring direct cell-cell communication via gap junctions between individual cell pairs,” Appl. Phys. Lett., vol. 86, pp. 3902, 2005
[7] F. K. Balagadde, L. You, C. L. Hansen, F. H. Arnold and S. R. Quake, “Long-term monitoring of bacteria undergoing programmed population control in a microchemostat,” Science, vol. 309, pp. 137-140, 2005
[8] M. P. MacDonald, G. C. Spalding and K. Dholakia, Microfluidic sorting in an optical lattice, Nature, vol. 426, pp. 421-424, 2003
[9] Y. Rondelez, G. Tresset, K. V. Tabata, H. Arata, H. Fujita, S. Takeuchi and H. Noji, Microfabricated arrays of femtoliter chambers allow single molecule enzymology, Nat. Biotechnol., vol. 23, pp. 361-365, 2005
[10] C. Ionescu-Zanetti, R. M. Shaw, J. Seo, Y. N. Jan, L. Y. Jan and L. P. Lee, “Mammalian electrophysiology on a microfluidic platform,” Proc. Natl. Acad. Sci. U.S.A., vol. 102, pp. 9112-9117, 2005
[11] T. Thorsen, S. J. Maerkl and S. R. Quake, “Microfluidic large-scale integration,” Science, vol. 298, pp. 580-584, 2002,
[12] Y. Li, Z. Wang, L. M. L. Ou, and H. Yu, “DNA detection on plastic: surface activation protocol to convert polycarbonate substrates to biochip platforms,” Anal. Chem., vol. 79, no. 2, pp. 426-433, 2007.
[13] Y. Li, L. M. L. Ou, and H. Yu, “Digitized molecular diagnostics: reading disk-based bioassays with standard computer drives,” Anal. Chem., vol. 80, no. 21, pp. 8216-8223, November 2008.
[14] J. J. La Clair and M. D. Burkat, “Molecular screening on a compact disc,” Org. Biomol. Chem., vol. 1, pp. 3244-3249, August 2003.
[15] S. Morais, J. Carrascosa, D. Mira, R. Puchades, and A. Maquieira, “Microimmunoanalysis on standard compact discs to determine low abundant compounds,” Anal. Chem., vol. 79, no. 20, pp. 7628-7635, October 2007.
[16] S. Lai, S. Wang, J. Luo, L. J. Lee, S. Yang, and M. J. Madou, “Design of a compact disk-like microfluidic platform for enzyme-linked immunosorbent assay,” Anal. Chem., vol. 76, no. 7, pp. 1832-1837, February 2004.
[17] J. Ducree, S. Haeberle, S. Lutz, S. Pausch, F. von Stetten, and R. Zengerle, “The centrifugal microfluidic bio-disk platform,” J. Micromech. Microeng., vol. 17, pp. 103-115, June 2007.

What is claimed:

1. A health diagnostic compact disc (HDCD) comprising:
(a) a protective layer;
(b) a reflective layer;
(c) a dye layer
(d) a polycarbonate layer; and
(e) a microfluidic layer.

2. The HDCD of claim 1, wherein the polycarbonate layer contains a data layer.

3. The HDCD of claim 1, wherein the microfluidic layer is composed of polydimethylsiloxane (PDMS).

4. The HDCD of claim 1, wherein the microfluidic layer contains one or more donut-shaped trenches.

5. The HDCD of claim 4, further comprising the microfluidic layer associated with conjugated microparticles in solution.

6. The HDCD of claim 1, further comprising integrated microneedles.

7. A method for detecting the presence of biomolecules or cells comprising:
(a) loading the microfluidic layer of the HDCD of claim 1 with conjugated microparticles in solution;
(b) incubating the HDCD to allow for microparticles to immobilize to the polycarbonate layer;
(c) using a standard compact disc (CD) drive to establish a baseline reading;
(d) loading a biomolecule or cell solution to the into the microfluidic layer of the HDCD;
(e) incubating the HDCD to allow for interaction of the biomolecule or cell solution with the immobilized microparticles;
(f) using a standard CD drive to obtain error rates; and
(e) comparing the data generated in steps (c) and (f) for determining a diagnosis.

8. The method of claim 7, wherein the diagnosis comprises cell counting, detecting biomolecule or cell concentration, detecting cell type, or detecting biomolecular binding.

9. The method of claim 8, wherein the biomolecular binding is an ELISA.

10. The method of claim 8, wherein the cell counting comprises counting of red blood cells for diagnosing leukemia, anemia, Gibson syndrome, COPD, sickle cell disease, internal bleeding, fever, chronic inflammation, heart disease, heart attack, myoinfarction, issues related to liver function, coronary heart disease, septics or acquired immunodeficiency disorder (AIDS).

11. The method of claim 8, wherein the cell counting comprises counting of red blood cells for determining effectiveness of chemotherapy, athletic ability or athletic stamina.

12. A kit for detection of biomolecules or cells comprising the HDCD of claim 1 and software for use in a personal computer which generates a diagnostic report of the detected biomolecules or cells.
